(19) United States
(12) Patent Application Publication
(10) Pub. No.: US 2008/0294219 A1
(43) Pub. Date: Nov. 27, 2008

(54) Power Management System for an Implantable Medical Device

(76) Inventors: Thomas P. Osypka, Palm Harbor, FL (US); Nicholas Vidalis, Clearwater, FL (US)

Correspondence Address: Edwards Angell Palmer & Dodge LLP, P.O. Box 55874, Boston, MA, 02205, US

(21) Appl. No.: 12/125,805

(22) Filed: May 22, 2008

Related U.S. Application Data
(60) Provisional application No. 60/931,251, filed on May 22, 2007.

(51) Int. Cl. A61N 1/00

(52) U.S. Cl. 607/32; 607/60

(57) ABSTRACT

An implantable medical and related cardiac rhythm management system include a device having a housing, means disposed within the housing for performing a function, and a header operatively associated with the housing, containing a first coil communicating with the means for facilitating a function, wherein the first coil is adapted and configured to facilitate bi-directional inductive telemetry with a second coil associated with an external programming device. Alternatively, in accordance with another aspect of the invention, the external programming device includes a second coil adapted and configured to form an inductive link with the first coil to facilitate uni-directional inductive telemetry between the external programming device and the pacemaker, with return communications being transmitted separately from the pacemaker to the external programming device.

### CROSS-REFERENCE TO RELATED APPLICATIONS

This application claims benefit of priority of U.S. Patent Application Ser. No. 60/931,251, filed May 22, 2007, which application is hereby incorporated by reference in its entirety.

### BACKGROUND OF THE INVENTION

1. Field of the Invention

The subject invention is directed to a system for managing power in an implantable medical device and more particularly to a system for facilitating bidirectional telemetry in an implantable medical device, such as, for example, an implantable cardiac rhythm management device through an inductive data link and for harvesting power from the inductive link to perform extended interrogation of the device.

2. Description of Related Art

It is well known to implant medical devices in human bodies to monitor physiological conditions, diagnose diseases, treat diseases or restore functions of organs or tissues. Examples of such implantable medical devices include cardiac rhythm management systems, neurological stimulators and neuromuscular stimulators, all of which may be embodied by the subject invention. Because these devices may be implanted in a patient for extended period of time, the size and power consumption of the devices are inherently constrained. Consequently, an implantable medical device may depend on an external system to perform certain functions.

Communication between an implantable medical device and an external system is referred to as telemetry. Examples of telemetry functions include programming the implantable device to perform certain monitoring or therapeutic tasks, extracting an operational status of the implantable device, transmitting real-time physiological data acquired by the implantable device, and extracting physiological data acquired by and stored in the implantable device.

Cardiac rhythm management devices are implanted in a patient to treat irregular or other abnormal cardiac rhythms by delivering electrical pulses to the patient's heart. Implantable cardiac rhythm management devices include, among other things, pacemakers. Pacemakers are often used to treat patients with slow or irregular heartbeats. Such pacemakers may coordinate atrial and ventricular contractions to improve the heart's pumping efficiency. Implantable cardiac management devices also include defibrillators that are capable of delivering higher energy electrical stimuli to the heart. Defibrillators may also include cardioverters, which synchronize the delivery of such stimuli to portions of sensed intrinsic heart activity signals. Defibrillators are often used to treat patients with heartbeats that are too fast.

An implantable cardiac rhythm management device typically communicates with an external device referred to as a programmer by way of telemetry. One type of telemetry is based on inductive coupling between two closely-placed coils using the mutual inductance between these coils. This type of telemetry is referred to as inductive telemetry or near-field telemetry, because the coils must be closely situated for obtaining inductively coupled communication.

In one example of inductive telemetry, an implantable device includes a first coil and a telemetry circuit, both sealed in a metal housing or “can,” which is typically made from titanium. The external programmer provides a second coil in a wand that is electrically connected to the programmer. During device implantation, a physician can evaluate a patient's condition, such as by using the implanted device to acquire real-time physiological data from the patient and communicating the physiological data in real-time to the external programmer for processing and/or display. The physician may also program the implantable device, including selecting a pacing or defibrillation therapy mode, and parameters required by that mode, based on the patient's condition and needs. The data acquisition and device programming are both performed using the inductive telemetry.

However, during inductive telemetry a significant amount of power is required for the first coil of the telemetry circuit, which is sealed in a metal housing, to communicate with the second coil provided in an external wand. Furthermore, Applicants recognize that performing a diagnostic program on a patient during an office visit, such as an electrocardiogram, will require the microprocessor within the implanted pacemaker device to utilize a relatively large amount of power from the internal battery. If repeated over time, such procedures will significantly reduce the useful life of the battery.

It would be beneficial therefore to provide an implantable medical device designed to facilitate bi-directional inductive telemetry in a manner that advantageously conserves the power stored in a battery, thus increasing the performance and longevity of the device.

### SUMMARY

The subject invention is directed to a new and useful system for an implantable cardiac rhythm management device, which employs inductive coupling in the form of two air core coils, similar to a split transformer. The inductive link between the two coils is used as a carrier wave to transmit data between an external programmer and the implanted device. At the base station, data is modulated and superimposed on the carrier wave. The data is then demodulated at the implanted device. Data can also be modulated at the implant and demodulated at the base station. Thus, bidirectional telemetry is made possible by the inductive link. In addition, power is tapped off or harvested from the implanted device coil, or “first coil,” through the inductive link.

In accordance with the subject invention, the primary coil winding of the transformer is housed within the header of the device, which can be an artificial pacemaker, for example. This advantageously helps shield the pacemaker circuitry from the electromagnetic field, allows transmission of auxiliary power into the implant for extended interrogation time where normally there would be insufficient power to do so, and allows for the implementation of bidirectional telemetry.

In accordance with the invention, the first coil can be coiled in the plane of the implantable medical device. In any case, the first coil can be arranged in the header to inductively interface with a magnetic field oriented perpendicularly to the plane of the medical device. Accordingly, if the device is oriented so that the major plane of the device is parallel to the nearby surface of the patient's body, the first coil is therefore arranged in the header to inductively interface with a magnetic field oriented substantially perpendicularly to the surface of a patient's body. However, if the device must be implanted in a different orientation, the coil can be arranged in a different position in the header, so that an effective inductive link can be established between the device and an external programmer and/or power source.

These and other features of the system of the subject invention will become more readily apparent to those having ordinary skill in the art from the following enabling description of the preferred embodiments of the subject invention taken in conjunction with the several drawings described below.

### BRIEF DESCRIPTION

So that those skilled in the art to which the subject invention appertains will readily understand how to make and use the bi-directional inductive telemetry and power harvesting arrangement of the subject invention without undue experimentation, preferred embodiments thereof will be described in detail hereinbelow with reference to certain figures, wherein:

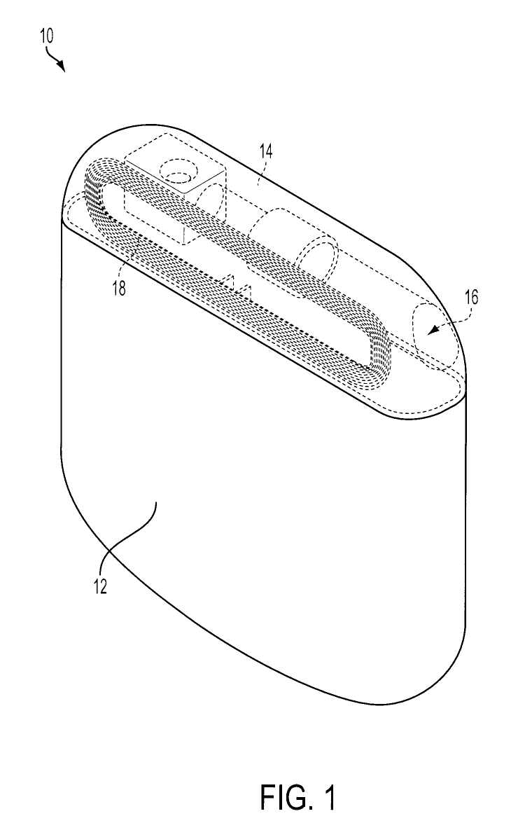

FIG. 1 is an illustration of an implantable cardiac rhythm management device or pacemaker having a header cavity which houses a coil of a telemetry circuit used to facilitate communication between the implanted device and an external programmer;

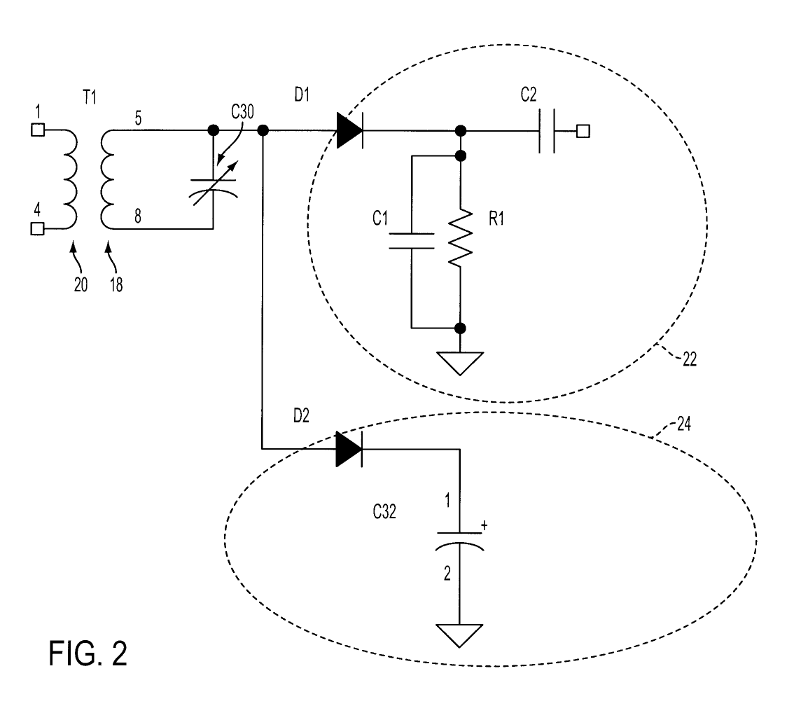

FIG. 2 is a schematic diagram illustrating the inductive coils of the air core transformer and the associated modulation/demodulation and power harvesting circuitry in accordance with one aspect of the invention;

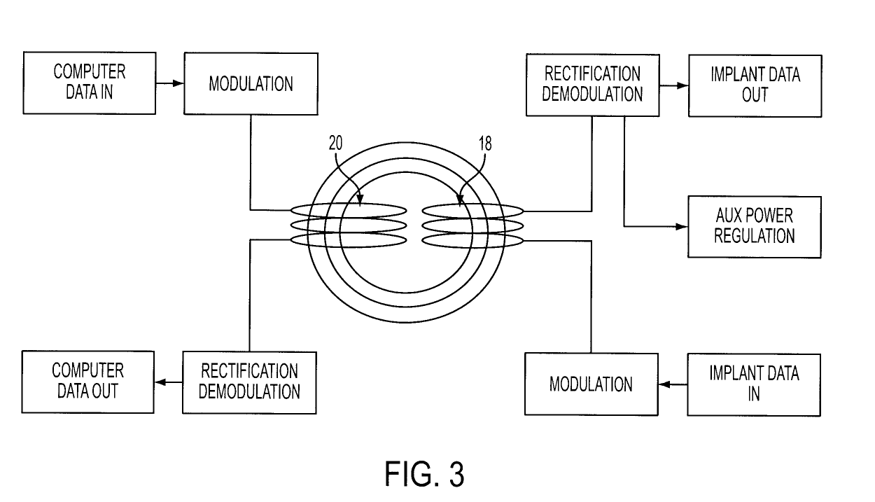

FIG. 3 is a block diagram illustrating the flow of data over a carrier wave facilitated by the bidirectional telemetry achieved in accordance with the embodiment of FIG. 2;

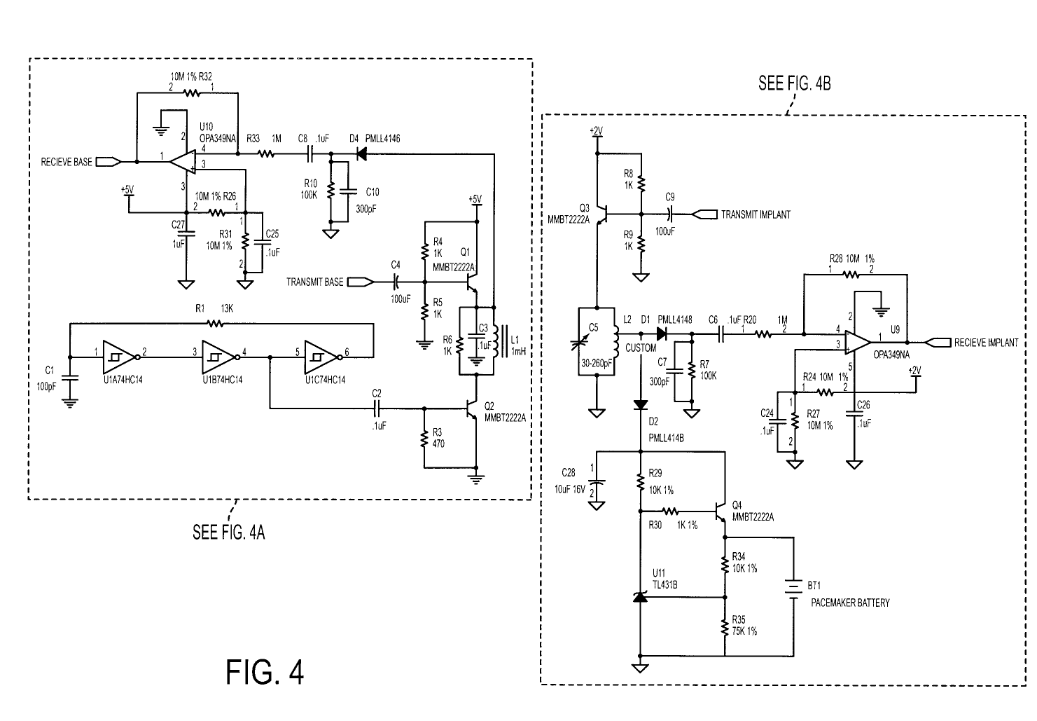

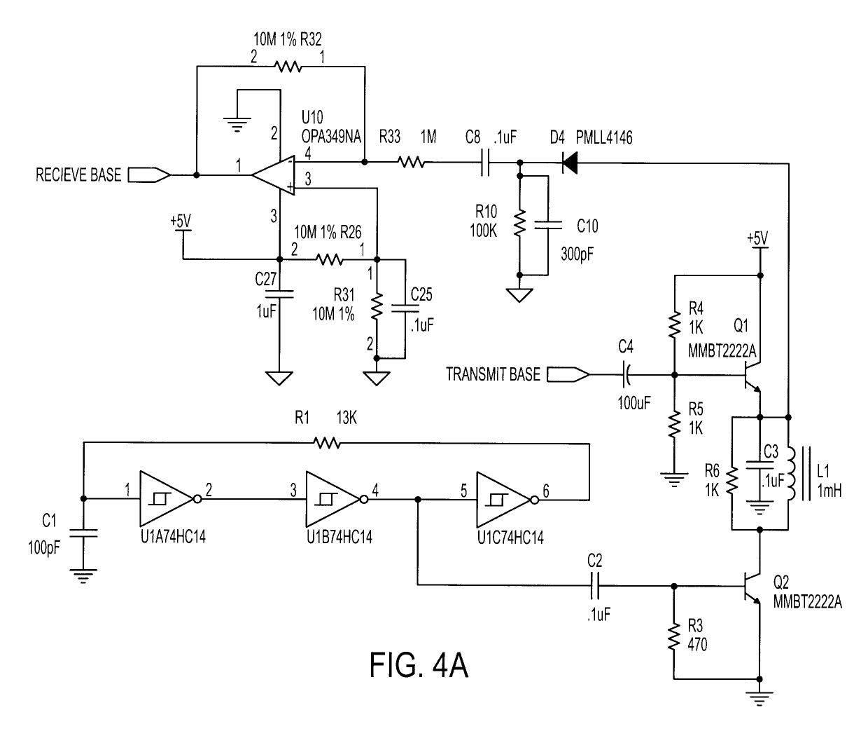

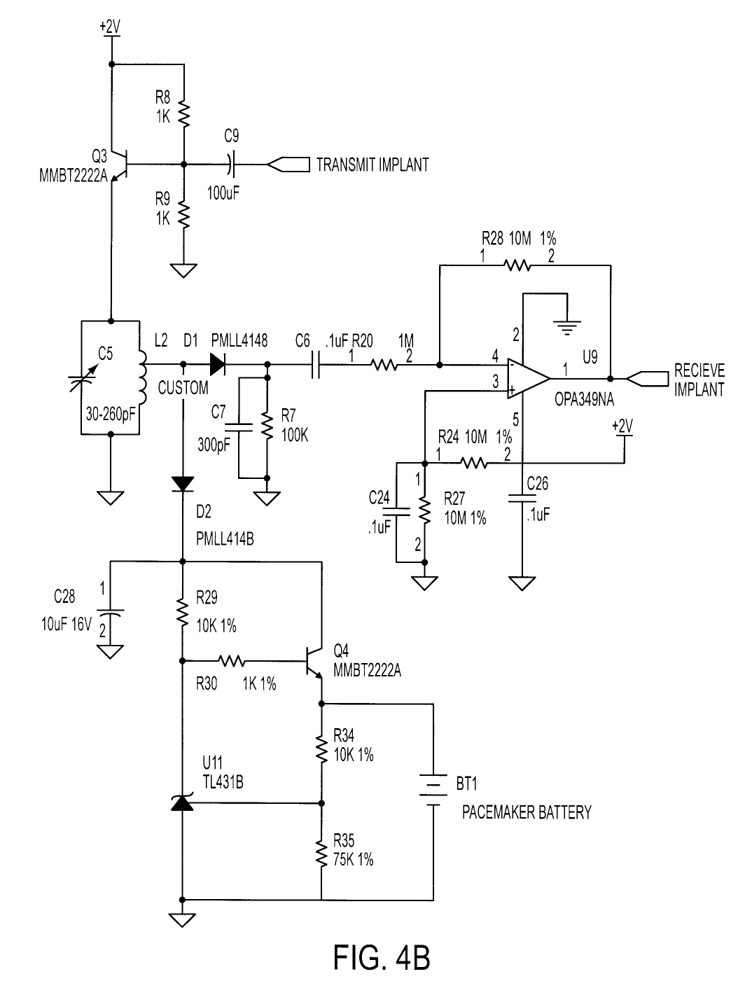

FIG. 4 is a more detailed schematic diagram of the circuitry for facilitating bidirectional telemetry and power harvesting in an implantable cardiac rhythm management device, in accordance with the embodiment of FIGS. 2 and 3;

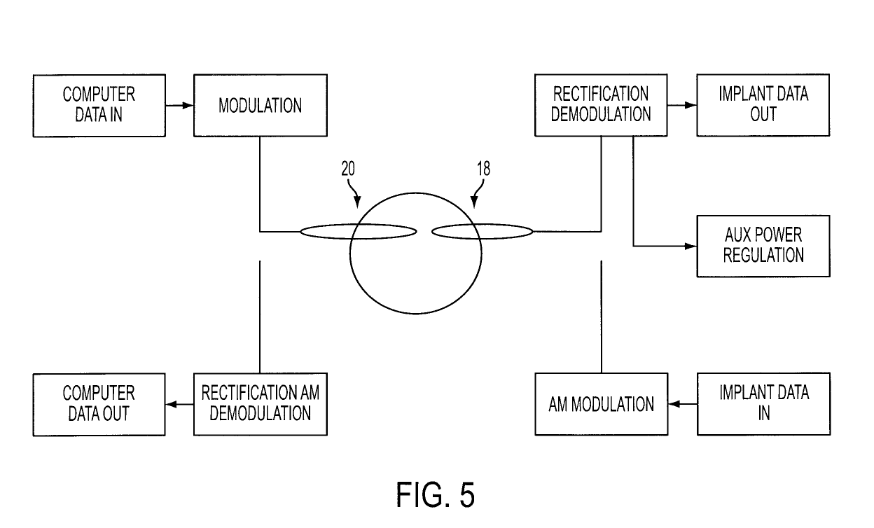

FIG. 5 is a more detailed schematic diagram of the circuitry for facilitating bidirectional telemetry and power harvesting in accordance with an alternative embodiment of the invention; and

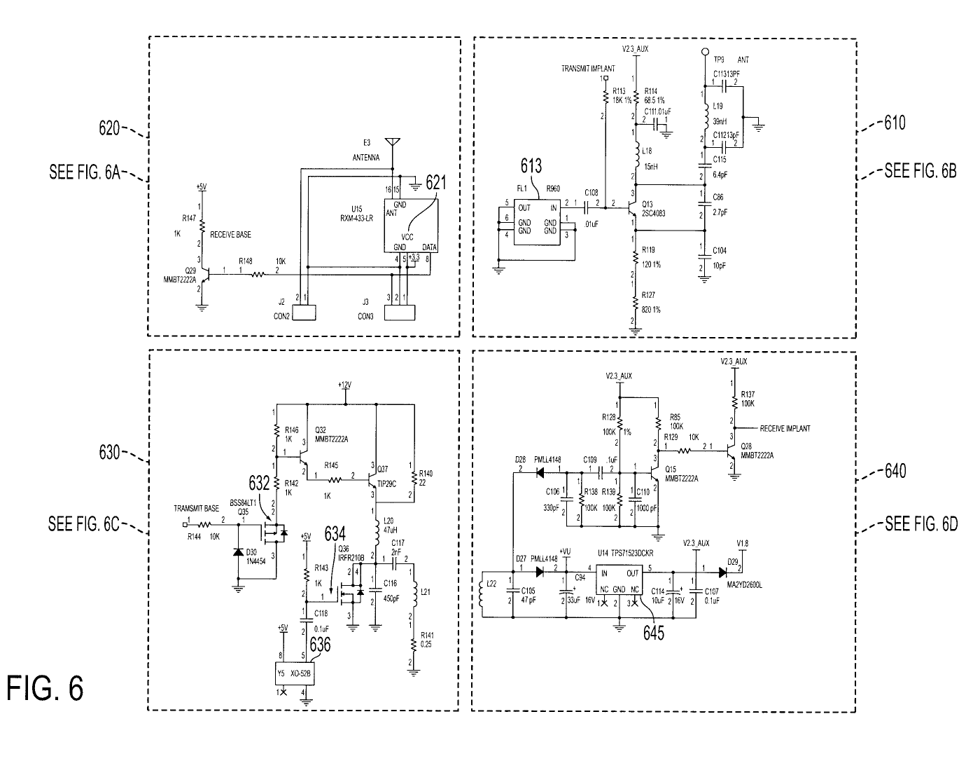

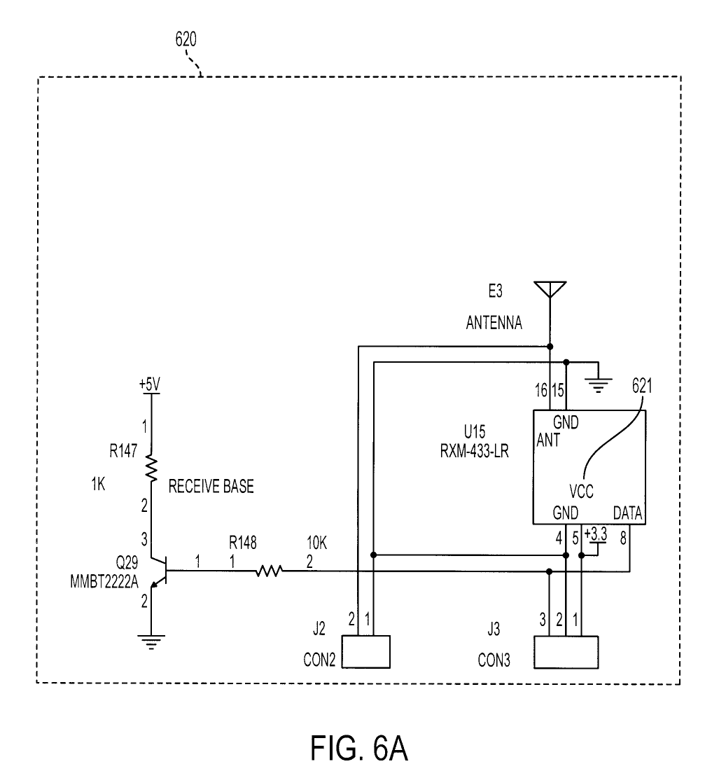

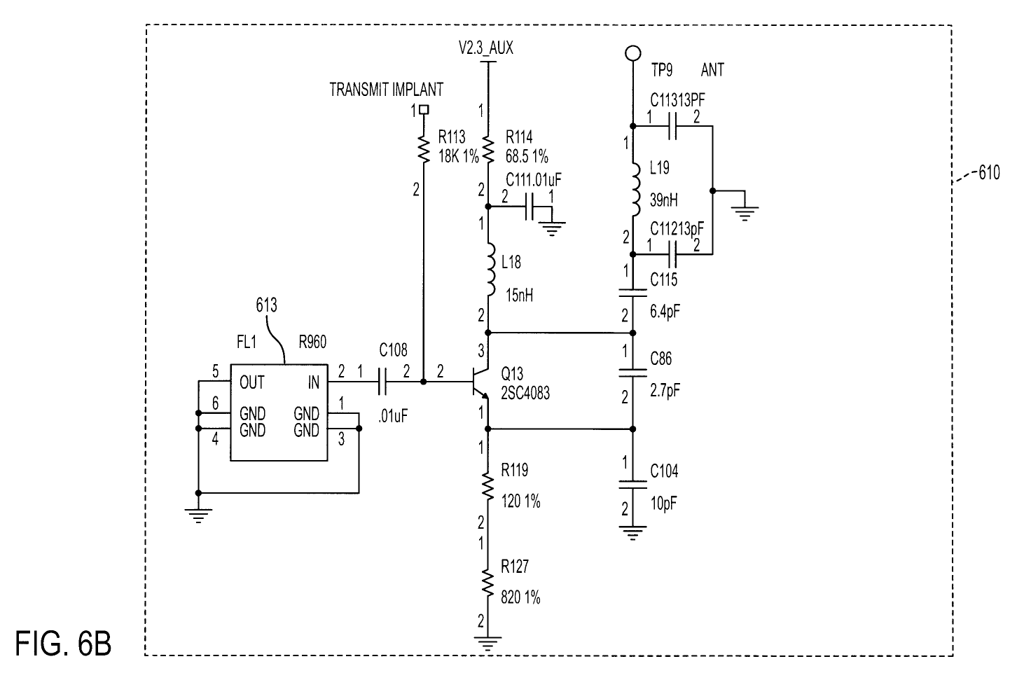

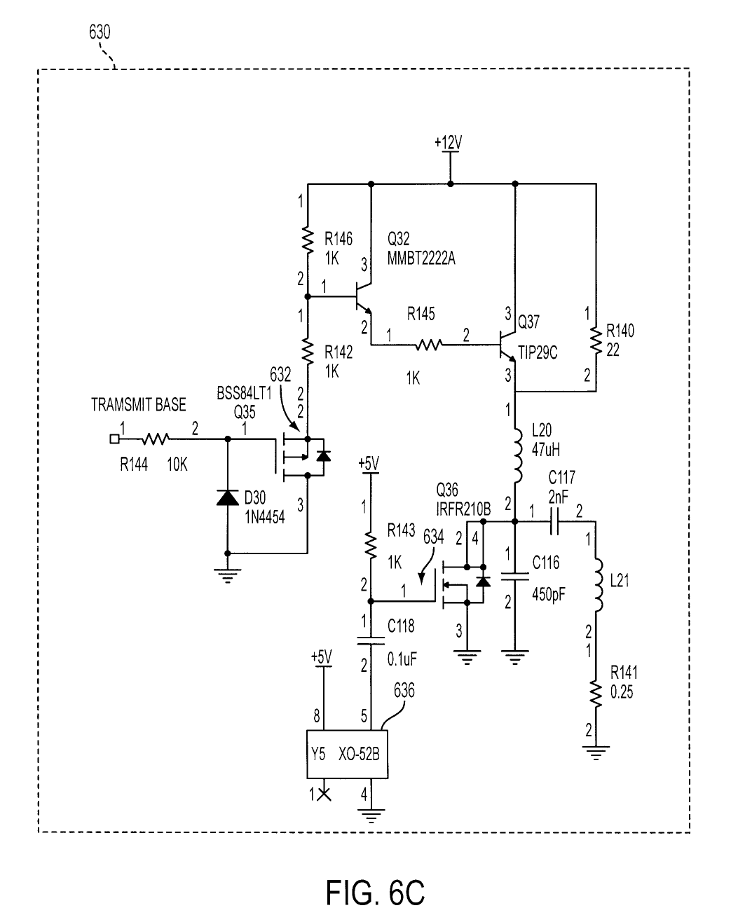

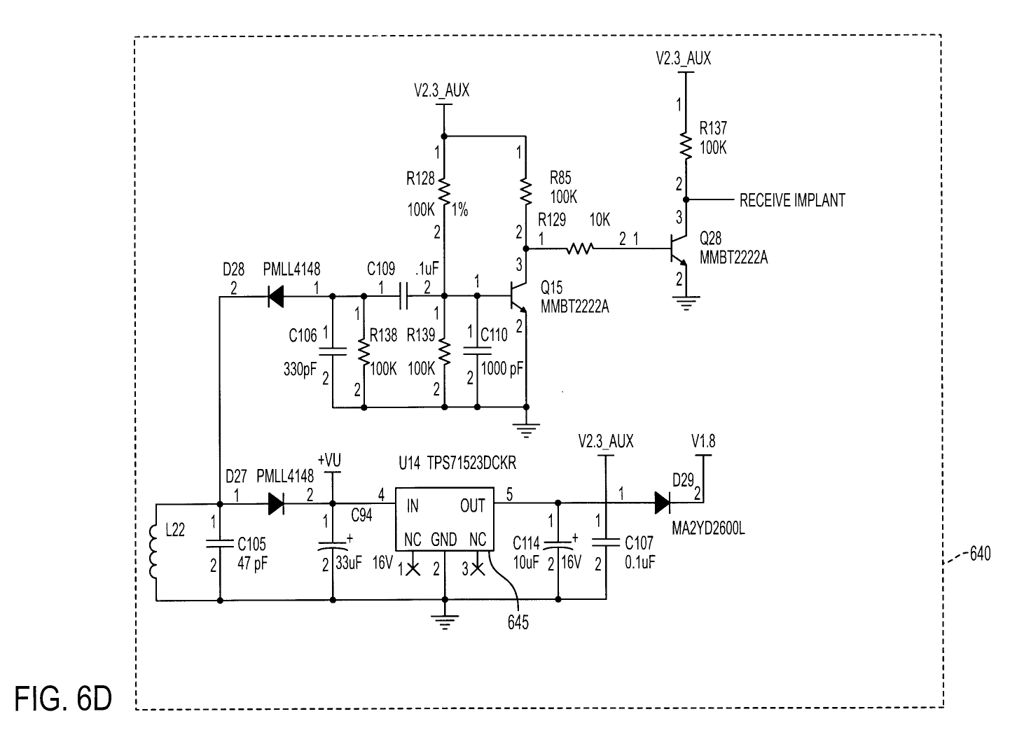

FIG. 6 is a block diagram of the embodiment of FIG. 4, illustrating unidirectional flow of data over a carrier wave used for power harvesting in an implant, with return communications via a respective implant AM transmitter and external AM receiver.

### DETAILED DESCRIPTION

Referring now to the drawings wherein like reference numerals identify similar structural features or aspects of the subject invention, there is illustrated in FIG. 1 an implantable medical device, and more particularly, a cardiac rhythm management device or pacemaker constructed in accordance with a preferred embodiment of the subject invention and designated generally by reference numeral 10. Pacemaker 10 includes a housing or “can” 12 constructed from a biocompatible metal, such as, for example, titanium, and housing, among other things, pacemaker circuitry and a battery (not shown).

Pacemaker 10 further includes a header 14 constructed from a bio-compatible thermoplastic material, such as, for example, a polycarbonate material. Header 14 defines a cavity that includes at lest one receptacle 16 for receiving the connector of an implantable cardiac lead, such as, for example, an IS-1 type connector associated with a cardiac pacing/sensing lead. In addition, the header 14 houses a first coil winding 18 that forms one half of an inductive coupling arrangement. By placing the coil 18 in the header 14, the pacemaker circuitry and electronics (e.g., a microprocessor) housed within the can of the pacemaker 12 are at least partially shielded from the electromagnetic field generated by the coil 18, during inductive coupling with a second coil winding located within a handheld wand connected to a serial port of an external programming device (see FIG. 3).

The coil 18, as illustrated in FIG. 1, can be arranged such that it is coiled in the plane of the pacemaker 10. This allows for an advantageous geometry for interfacing with the external coil, placing the strongest magnetic field perpendicular to the coil, and therefore minimizes the field passing through the housing 12 and the electronic components housed therein. The pacemaker 10 can therefore be implanted in the patient such that the coil 18 can easily inductively interface with an external device. Alternatively, if necessary, the coil can be arranged in a different orientation or configuration, such as by being coiled around the receptacle 16, for example.

In accordance with the invention, an external device wand includes a coil that is arranged so as to facilitate an inductive link between the external device and the implanted device.

Referring now to FIG. 3, the coil 18 provides for an inductive link with a second coil winding 20 associated with an external wand (not shown). Together, the two coils 18, constitute a transformer air core coil arrangement for facilitating bi-directional telemetry over a carrier wave between the external programming device and the circuitry housed within pacemaker 10.

As best seen in FIG. 2, coil 18 is operatively associated with a tunable capacitor C30 located within the pacemaker housing 12 for fine-tuning the coil 18 to a desired frequency for receiving/transmitting data. The tunable capacitor C30 is connected to a modulation/demodulation circuit 22 and a power harvesting circuit 24 located within the housing or can 12 of the pacemaker 10.

As illustrated in FIG. 4 for example, the output from demodulation circuit 22 is directed to the pacemaker circuitry to program the implantable device 10, including, for example, selecting a pacing or defibrillation therapy mode, and certain parameters required by that mode. The output from the power harvesting circuit 24 is directed to a regulator that regulates power from the coil 18 to the pacemaker battery, whereby the additional power can be used to supply the microprocessor of the pacemaker during the performance of a diagnostic task requiring extended interrogation, such as, for example, an electrocardiogram, which, in the absence of the additional power supplied by the secondary coil, would put a substantial drain of the battery, decreasing its longevity.

Referring again to FIG. 3, in accordance with one aspect of the subject invention, bi-directional telemetry is facilitated over a carrier wave transmitted between coil 18 in the pacemaker header 14 and coil 20 in a handheld wand associated with an external programming device. As shown, data from the external programming device is first modulated by the electronics associated therewith. That data is then superimposed on the carrier wave bridging the gap between coils 18 and 20. The data received at the pacemaker 10 is than rectified and demodulated and delivered to the implant circuitry. In addition, power transmitted through the inductive coupling of the two coils is conducted to the auxiliary power regulator for harvesting. In addition, the inductive link between coils 18 and 20 is utilized to transfer data from the implanted device 10 to the external programming device. That is, data is modulated by the pacemaker electronics and superimposed on a carrier wave bridging the gap between coils 18 and 20. That data is then rectified and demodulated by the external programming device.

In accordance with another aspect of the invention and as illustrated in FIGS. 5 and 6, forward communications and power for harvesting can be sent to the implant from an external device, such as a programming wand, via a transmit circuit 630 thereof, by superimposing data on a carrier wave, which in accordance with one aspect, has a frequency of about 1 MHz. This carrier wave can also be used to provide power to the implant through harvesting, as set forth above. The data and power are received by an implant receiving and power harvesting circuit 640. In accordance with this aspect, however, return communications from the implant are provided by a separate transmitter and receiver arranged respectively in the implant and external device. As illustrated in FIGS. 5 and 6, for example, and in accordance with one embodiment, an ultra low power AM transmitter circuit 610 is provided in the implant, which can operate at 433 MHz, for example. The AM transmitter circuit 610 can be powered by way of power harvesting through an auxiliary voltage regulator, for example. Accordingly, an AM receiver circuit 620 is provided in the external device, to receive the data sent by the implant.

As illustrated particularly in FIG. 6, the AM transmitter circuit 610 in the implant, as embodied, includes a surface acoustic wave (SAW) filter and front end amplifier 613 provided therein, in addition to standard componentry, as illustrated. It is to be noted that although in accordance with a preferred aspect, the AM transmitter circuit 610 operates at 433 MHz, the circuit can be adapted to operate at any desired frequency. Additionally, in the illustrated embodiment, specific values for circuit components, such as resistance, capacitance and inductance, are provided, as well as designations for specific diodes and transistors. However, it is to be understood that the present invention is not limited only to the specific values and components presented in the Figures. Depending on the implementation and application, values and types of circuit components can be selected accordingly. The AM receiver circuit 620, accordingly can include an appropriate receiver 621 for the transmitting frequency of the implant, in the illustrated embodiment, for a frequency of about 433 MHz.

The base transmitting circuit 630 can include any necessary componentry to carry out the desired functions, as set forth herein. As illustrated in FIG. 6, the base transmitting circuit 630 includes an oscillator 636, which in the preferred embodiment operates at about 1 MHz. Additionally, a main frequency power oscillator 634, and front end preamplifier 632 are provided, in addition to otherwise basic circuit componentry. As set forth above, it is to be understood that the present invention is not limited only to certain circuit components or only those having the specific values presented in the Figures.

The implant receiving and power harvesting circuit 640 includes an auxiliary voltage regulator 645 in addition to other basic circuit components, the specific values of which are not limited to those illustrated in the figures.

Naturally, although return communications in accordance with this latter aspect of the invention are described as being carried out by way of amplitude modulation, it is to be understood that any suitable integrated transmission system may be used alternatively, and still be in keeping with the present invention.

In sum, the subject invention, in one aspect, is directed to an implantable medical device that includes, among other things, a housing and means disposed within the housing for performing a function, such as, for example, cardiac rhythm management, defibrillation, hemo-dynamic monitoring or neuro-stimulation. A header is operatively associated with the housing and it contains a first coil that communicates with the means for performing a function. The first coil, in accordance with one aspect is adapted and configured to facilitate bi-directional inductive telemetry with a second coil associated with an external programming device. In accordance with another aspect, forward communications to the implant are carried over the first and second coils, while return or “back” communication from the implant to the external programming device are carried over a substantially independent transmitter and receiver pair. The implantable medical device further includes means disposed within the housing for harvesting energy from the first coil, and a power regulator disposed within the housing for regulating power harvested from the first coil.

The subject invention is also directed to a cardiac rhythm management system that includes, among other things, an implantable pacemaker having a housing with a header cavity. Means are disposed within the housing for facilitating cardiac rhythm management and a first coil is disposed within the header cavity, wherein the first coil communicates with the means for performing a function, such as facilitating cardiac rhythm management, as set forth hereinabove. The system in accordance with one aspect further includes an external programming device having a wand operatively associated therewith, wherein the wand includes a second coil adapted and configured to form an inductive link with the first coil to facilitate bi-directional inductive telemetry between the external programming device and the pacemaker. In accordance with another aspect of the invention, the wand includes a second coil adapted and configured to form an inductive link with the first coil to facilitate unidirectional inductive telemetry between the external programming device and the pacemaker, with return communications being transmitted separately from the pacemaker to the external programming device. The system further includes means disposed within the pacemaker housing for harvesting energy from the first coil.

While the apparatus and system of subject invention have been shown and described with reference to preferred embodiments, and in particular with reference to an implantable cardiac rhythm management device, those skilled in the art will readily appreciate that the subject invention can be extended to implantable devices for cardiac pacing, defibrillation, hemo-dynamic monitoring as well as and neuro-stimulation, and that changes and/or modifications may be made to the preferred embodiments of the invention without departing from the spirit and scope of the subject invention.

What is claimed is:

1. An implantable medical device comprising:
a) a housing;
b) means disposed within the housing for performing a function; and
c) a header operatively associated with the housing and containing a first coil communicating with the means for facilitating a function, wherein the first coil is adapted and configured to facilitate inductive telemetry with a second coil associated with an external programming device.

2. The implantable medical device as recited in claim 1, wherein the first coil is adapted and configured to facilitate bi-directional inductive telemetry with a second coil associated with an external programming device.

3. The implantable medical device as recited in claim 1, wherein the first coil is adapted and configured to facilitate uni-directional inductive telemetry with a second coil associated with an external programming device, to provide forward communication to the implantable medical device.

4. The implantable medical device as recited in claim 3, wherein return communications from the implantable medical device are transmitted though a secondary transmitter.

5. An implantable medical device as recited in claim 1, further comprising means disposed within the housing for harvesting energy from the first coil.

6. An implantable medical device as recited in claim 5, further comprising a power regulator disposed within the housing for regulating power harvested from the first coil.

7. An implantable medical device as recited in claim 1, wherein the means for performing a function includes means for further comprising a power regulator disposed within the housing for facilitating cardiac rhythm management.

8. An implantable medical device as recited in claim 1, wherein the first coil is coiled in the plane of the implantable medical device.

9. An implantable medical device as recited in claim 1, wherein the first coil is arranged in the header to inductively interface with a magnetic field oriented perpendicularly to the plane of the medical device.

10. An implantable medical device as recited in claim 1, wherein the first coil is arranged in the header to inductively interface with a magnetic field oriented substantially perpendicularly to the surface of a patient's body, when implanted therein.

11. A cardiac rhythm management system comprising:
a) an implantable pacemaker having a housing with a header cavity, wherein means are disposed within the housing for facilitating cardiac rhythm management and a first coil is disposed within the header cavity, wherein the first coil communicates with the means for facilitating cardiac rhythm management; and
b) an external programming device having a wand operatively associated therewith, wherein the wand includes a second coil adapted and configured to form an inductive link with the first coil to facilitate bidirectional inductive telemetry between the external programming device and the pacemaker.

12. A cardiac rhythm management system as recited in claim 11, further comprising means disposed within the pacemaker housing for harvesting energy from the first coil.

13. A cardiac rhythm management system as recited in claim 12, further comprising a power regulator disposed within the housing for regulating power harvested from the first coil.

14. A cardiac rhythm management system as recited in claim 11, wherein the first coil is arranged in the header and the second coil is arranged in the wand to facilitate an inductive link therebetween.

15. A cardiac rhythm management system as recited in claim 14, wherein the first coil and the second coil are arranged so as to generate parallel magnetic fields when in use.
